(19) United States
(12) Patent Application Publication
(10) Pub. No.: US 2014/0099706 A1
(43) Pub. Date: Apr. 10, 2014

(54) Photobioreactor With A Thermal System, And Methods of Using The Same

(71) Applicant: Joule Unlimited Technologies, Inc., Bedford, MA (US)

(72) Inventors: Michael Y. Leshchiner, Needham, MA (US); Stuart A. Jacobson, Lexington, MA (US)

(73) Assignee: Joule Unlimited Technologies, Inc., Bedford, MA (US)

(21) Appl. No.: 14/026,274

(22) Filed: Sep. 13, 2013

Related U.S. Application Data
(60) Provisional application No. 61/701,468, filed on Sep. 14, 2012.

(51) Int. Cl. C12M 1/00

(52) U.S. Cl. CPC C12M 21/02
USPC 435/292.1; 435/317.1

(57) ABSTRACT

The invention relates to a photobioreactor system for a phototrophic microorganism, and culture medium therefor, comprising a reactor chamber and a thermal system. The thermal system includes a convection chamber in thermal contact with the reactor chamber and having a first port and a second port; a heat storage reservoir having a first region containing a first volume of heat exchange liquid at a first temperature and a second region containing a second volume of heat exchange liquid at a second temperature; and a flow system configured for (1) flowing heat exchange liquid from the heat storage reservoir into the first port and through the convection chamber and flowing heat exchange liquid from the convection chamber out of the second port into the heat storage reservoir; and (2) flowing heat exchange liquid from the heat storage reservoir into the second port and through the convection chamber and flowing heat exchange liquid from the convection chamber out of the first port into the heat storage reservoir. Methods of using said photobioreactor to manage the temperature of the culture medium in the photobioreactor and to manage the amount of heat stored in the reservoir are also described.

### RELATED APPLICATION

This application claims the benefit of U.S. Provisional Application No. 61/701,468, filed on Sep. 14, 2012. The entire teachings of the above application are incorporated herein by reference.

### BACKGROUND OF THE INVENTION

Conventional approaches for thermal management of reactors rely on external heat exchangers in combination with thermal sinks, such as evaporative cooling towers, ground water, rivers, or oceans, to dissipate heat. Such cooling schemes are most economical when the thermal load is relatively constant on a daily and year-round basis, as is the case in many industrial systems and processes.

However, photobioreactors operate by capturing solar radiation, which is highly cyclical and has sharp daily peaks and substantial seasonal variation. As such, to be functional, the thermal management system of a photobioreactor must be sized for the worst possible conditions observed during the year at the particular location, a practice that results in over-sized equipment that is under-utilized for much of the year. The nature of solar radiation is also such that the sharp daily peaks, which necessitate high power consumption in order to effect heat rejection, coincide with the worst ambient conditions (e.g., highest temperatures) of the day, increasing the challenge of heat rejection. Furthermore, thermal management systems for photobioreactors consume large amounts of water, since evaporative cooling is often the best heat rejection technique in locations with favorable yearly amounts of solar radiation, where daily temperatures can exceed 40° C.

Therefore, there is a need for a thermal management system for photobioreactors having reduced power and/or water consumption that operates efficiently in places that experience high thermal loads, sharp daily peaks and substantial seasonal variation of solar radiation.

### SUMMARY OF THE INVENTION

The invention relates, in one aspect, to a photobioreactor system comprising a thermal management system provided, at least in part, by a heat storage reservoir having a first region containing a first volume of heat exchange liquid at a first temperature and a second region containing a second volume of heat exchange liquid at a second temperature. Further aspects of the invention include methods of using said photobioreactor system to manage the temperature of the culture medium in the photobioreactor and to manage the amount of heat stored in the reservoir.

One embodiment of the invention is a photobioreactor system for a phototrophic microorganism, and culture medium therefor, comprising a reactor chamber for enclosing the phototrophic microorganism and culture medium therefor, the reactor chamber being at least partially transparent for light of a wavelength that is photosynthetically active in the phototrophic microorganism; and a thermal system. The thermal system comprises a convection chamber in thermal contact with the reactor chamber and having a first port and a second port; a heat storage reservoir having a first region containing a first volume of heat exchange liquid at a first temperature and a second region containing a second volume of heat exchange liquid at a second temperature; and a flow system configured for (1) flowing heat exchange liquid from the heat storage reservoir into the first port through the convection chamber and flowing heat exchange liquid from the convection chamber out of the second port into the heat storage reservoir, and (2) flowing heat exchange liquid from the heat storage reservoir into the second port and through the convection chamber and flowing heat exchange liquid from the convection chamber out of the first port into the heat storage reservoir. The second temperature is greater than the first temperature.

Another embodiment of the invention is a method for managing the temperature of culture medium for a phototrophic microorganism in a photobioreactor system, the method comprising providing the photobioreactor system described above; and flowing heat exchange liquid from the first or second region of the heat storage reservoir through the flow system into the convection chamber and flowing heat exchange liquid from the convection chamber through the flow system and into the heat storage reservoir, thereby managing the temperature of the culture medium.

Yet another embodiment of the invention is a photobioreactor system for a phototrophic microorganism, and culture medium therefor, comprising a reactor chamber for enclosing the phototrophic microorganism and culture medium therefor, the reactor chamber being at least partially transparent for light of a wavelength that is photosynthetically active in the phototrophic microorganism and comprising a plurality of adjacent channels having substantially longer lengths than widths; and a thermal system. The thermal system comprises a convection chamber in thermal contact with the reactor chamber and having a first port and a second port; a heat storage reservoir abutting the convection chamber and having a first region containing a first volume of heat exchange liquid at a first temperature and a second region containing a second volume of heat exchange liquid at a second temperature; and a flow system configured for (1) flowing heat exchange liquid from the heat storage reservoir into the first port through the convection chamber and flowing heat exchange liquid from the convection chamber out of the second port into the heat storage reservoir, and (2) flowing heat exchange liquid from the heat storage reservoir into the second port and through the convection chamber and flowing heat exchange liquid from the convection chamber out of the first port into the heat storage reservoir, wherein the reactor chamber, the convection chamber, and the heat storage reservoir are separate chambers of a flexible polymeric capsule having a length of at least 50 m; and the first and second regions are connected by a liquid flow path containing thermally stratified heat exchange liquid that increases from the first temperature to the second temperature.

The inclusion of a thermal system as described herein in a photobioreactor system allows tight control of temperature variation inside the reactor chamber of the photobioreactor, resulting in tight control of the temperature of the culture medium therein and improved organism productivity. In addition, a substantial fraction of the energy stored in the reservoir can be used to maintain the temperature of the culture medium during non-productive hours and provide freeze protection, thereby extending productive hours. By storing heat in a heat storage reservoir (and particularly a stratified heat storage reservoir) of a thermal system described herein, heat rejection can be postponed until a more favorable time of the day/night, or can be spread out over a longer time period, significantly reducing the size and cost of the cooling equipment.

To summarize, the inclusion of a thermal system described herein in a photobioreactor system avoids using peaking power plants and instead shifts the burden to base load stations. Moreover, allowing the cooling system to operate overnight, instead of during peak daylight hours, could allow for the use of non-evaporative cooling methods, thus potentially eliminating water consumption or, at the very least, substantially reducing water consumption. In addition, by shifting a substantial portion of heat rejection to nighttime, the bioreactor itself can be used to radiate part of the heat load to the environment, allowing for a further decrease in the size of the cooling equipment.

### BRIEF DESCRIPTION OF THE FIGURES

The foregoing will be apparent from the following more particular description of example embodiments of the invention, as illustrated in the accompanying drawings in which like reference characters refer to the same parts throughout the different views. The drawings are not necessarily to scale, emphasis instead being placed upon illustrating embodiments of the present invention.

FIG. 1 is a diagram and shows an exemplary photobioreactor system of the invention.

FIG. 2 is a diagram illustrating the primary modes of operation of a photobioreactor system employing a thermal reservoir containing horizontally, thermally stratified heat exchange liquid.

FIG. 3A is a cross-sectional diagram and shows an exemplary photobioreactor of the invention.

FIG. 3B is a cross-sectional diagram and shows an exemplary photobioreactor of the invention.

FIG. 4 is a diagram and shows the piping and instrumentation of a test apparatus for evaluation of heat transfer between a reactor chamber and a convection chamber that share a common wall.

FIG. 5 is a graph and shows temperature as a function of time, as measured at four locations in the test apparatus depicted in FIG. 4.

FIG. 6 is a graph and shows the heat transfer coefficient as a function of Reynolds number, as measured in the test apparatus depicted in FIG. 4.

### DETAILED DESCRIPTION OF THE INVENTION

A description of example embodiments of the invention follows.

One embodiment of the invention is a photobioreactor system for a phototrophic microorganism, and culture medium therefor, comprising a reactor chamber for enclosing the phototrophic microorganism and culture medium therefor, the reactor chamber being at least partially transparent for light of a wavelength that is photosynthetically active in the phototrophic microorganism; and a thermal system. The thermal system comprises a convection chamber in thermal contact with the reactor chamber and having a first port and a second port; a heat storage reservoir having a first region containing a first volume of heat exchange liquid at a first temperature and a second region containing a second volume of heat exchange liquid at a second temperature; and a flow system configured for (1) flowing heat exchange liquid from the heat storage reservoir into the first port through the convection chamber and flowing heat exchange liquid from the convection chamber out of the second port into the heat storage reservoir, and (2) flowing heat exchange liquid from the heat storage reservoir into the second port and through the convection chamber and flowing heat exchange liquid from the convection chamber out of the first port into the heat storage reservoir.

Phototrophic microorganisms contained in photobioreactors require light for their growth and/or the production of carbon-based products of interest. Therefore, the photobioreactors, and, in particular, the reactor chambers are adapted to allow light of a wavelength that is photosynthetically active in the phototrophic microorganism to reach the culture medium and the phototrophic microorganism. Typically, the reactor chamber, or at least a portion thereof, is at least partially transparent for light of a wavelength that is photosynthetically active in the phototrophic microorganism. This can be achieved by forming the reactor chamber from a material, for example, thin-film polymeric material that is at least partially transparent for light of a wavelength that is photosynthetically active in the phototrophic microorganism.

As used herein, “light of a wavelength that is photosynthetically active in the phototrophic microorganism” refers to light that can be utilized by a phototrophic microorganism to grow and/or produce carbon-based products of interest, for example, fuels, including biofuels.

“Biofuel” refers to any fuel that is derived from a biological source, including one or more hydrocarbons, one or more alcohols, one or more fatty esters, or a mixture thereof. Ethanol or other liquid hydrocarbon fuels can be produced by phototrophic microorganisms.

“Carbon-based products of interest” include alcohols such as ethanol, propanol, isopropanol, butanol, fatty alcohols, fatty acid esters, ethyl esters, wax esters; hydrocarbons and alkanes such as propane, octane, diesel, Jet Propellant 8 (JP8); polymers such as terephthalate, 1,3-propanediol, 1,4-butanediol, polyols, Polyhydroxyalkanoates (PHA), poly-beta-hydroxybutyrate (PHB), acrylate, adipic acid, ε-caprolactone, isoprene, caprolactam, rubber; commodity chemicals such as lactate, docosahexaenoic acid (DHA), 3-hydroxypropionate, γ-valerolactone, lysine, serine, aspartate, aspartic acid, sorbitol, ascorbate, ascorbic acid, isopentenol, lanosterol, omega-3 DHA, lycopene, itaconate, 1,3-butadiene, ethylene, propylene, succinate, citrate, citric acid, glutamate, malate, 3-hydroxypropionic acid (HPA), lactic acid, THF, gamma butyrolactone, pyrrolidones, hydroxybutyrate, glutamic acid, levulinic acid, acrylic acid, malonic acid; specialty chemicals such as carotenoids, isoprenoids, itaconic acid; pharmaceuticals and pharmaceutical intermediates such as 7-aminodeacetoxycephalosporanic acid (7-ADCA)/cephalosporin, erythromycin, polyketides, statins, paclitaxel, docetaxel, terpenes, peptides, steroids, omega fatty acids and other such suitable products of interest. Such products are useful in the context of biofuels, industrial and specialty chemicals, as intermediates used to make additional products, such as nutritional supplements, nutraceuticals, polymers, paraffin replacements, personal care products and pharmaceuticals.

Typically, the reactor chamber(s) of the photobioreactor are adapted to allow cultivation of the phototrophic microorganisms in a thin layer. In some embodiments, the layer is between about 5 mm and about 30 mm thick, more typically, between about 20 mm and about 30 mm thick.

In some embodiments, the reactor chamber is divided widthwise into a plurality of adjacent channels positioned in parallel and having substantially longer lengths than widths.

Typically, the convection chamber provides convective heat exchange with culture medium in the reactor chamber by being in thermal contact with the reactor chamber. However, as will be discussed in further detail below, the convection chamber can also be filled with gas, thereby acting as an insulating barrier.

A reactor chamber and a convection chamber should be in thermal contact with one another over an area sufficient to facilitate managing the temperature of the culture medium in the reactor chamber. In preferred embodiments, the area over which the reactor chamber and the convection chamber are in contact, or the contact area, is at least a substantial portion of the area obtained by multiplying the length of the reactor chamber by the width of the reactor chamber. Thus, the reactor chamber and the convection chamber can be of substantially equal length and of substantially equal width. In a particular aspect of this embodiment, the length of the reactor chamber and the convection chamber is greater than 30 m. In another particular aspect of this embodiment, the length of the reactor chamber and the convection chamber is greater than 30 m and the width of the reactor chamber and the convection chamber is about 1 to about 3 m.

“Thermal system,” as used herein, refers to the combination of at least a convection chamber, a heat storage reservoir and a flow system configured for flowing heat exchange liquid from the heat storage reservoir through the convection chamber and flowing heat exchange liquid from the convection chamber into the heat storage reservoir. Although heat exchange liquid can be added to or removed from the thermal system, typically, the system is closed (e.g., not open to the environment). Typically, this means that the same volume of heat exchange liquid is recycled throughout operation of the thermal system. A thermal system can further include, for example, a heat rejection system.

The flow system (e.g., piping, tubing) can be configured to allow fluid communication between the thermal system and a liquid source (e.g., ocean, pond) to supply liquid to the system when additional heat exchange liquid is needed or when it is necessary to decrease the volume of heat exchange liquid in the system.

The heat storage reservoir stores heat in the form of heat exchange liquid. Typically, the heat exchange liquid is unreactive and is predominantly in liquid phase under the conditions maintained within the thermal system. Typically, the heat exchange liquid increases in temperature when it takes up heat and decreases in temperature when it rejects heat. The heat storage reservoir has a first region containing a first volume of heat exchange liquid at a first temperature and a second region containing a second volume of heat exchange liquid at a second temperature. The first region can be a first tank or container and the second region can be a second tank or container, wherein the two tanks or containers are configured for fluid communication via the convection chamber, but are not in direct fluid communication.

In other embodiments in which the first region and the second region are not in direct fluid communication, the first region and the second region are contained within a single tank or container comprising a flexible sheet that separates the first region from the second region. The flexible sheet separating the first region from the second region can flex or stretch, thereby providing increased capacity in, for example, the first region, as needed. Because the first and second regions are contained within a single tank or container in this embodiment, increasing the capacity in the first region also decreases the capacity in the second region. By using a flexible sheet to separate the first and second regions in a single tank or container, the total volume of the heat storage reservoir can be reduced.

Alternatively, the first and second regions are connected by a liquid flow path (e.g., are in direct fluid communication).

The heat storage reservoir can be a stratified heat storage reservoir, that is, the first and second regions are connected by a liquid flow path containing thermally stratified heat exchange liquid that increases from a first temperature to a second temperature. Typically, the stratified heat storage reservoir is designed such that no significant heat exchange between (1) the region between the first and second regions and (2) the outside environment occurs.

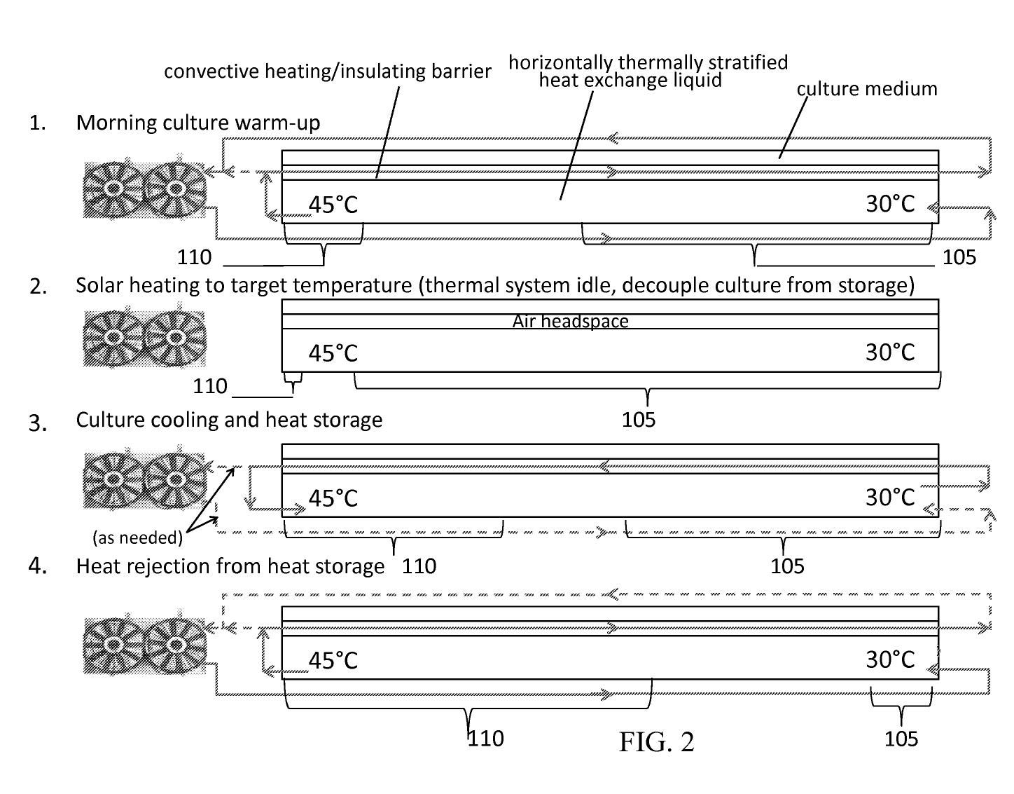

As used herein, “thermally stratified heat exchange liquid,” refers to a single volume of liquid contained in a container, such as a reservoir, wherein the liquid has a temperature that increases from a first temperature at the first region of the container (e.g., 105 in FIG. 2) to a second temperature at the second region of the container (e.g., 110 in FIG. 2).

In a particular embodiment, the thermally stratified heat exchange liquid is horizontally thermally stratified heat exchange liquid. The term “horizontal,” as used herein, is defined with respect to the surface which supports the reactor chamber (e.g., the ground, the water). Thus, “horizontal” is essentially parallel to the surface that supports the reactor chamber. If a reactor chamber is supported by an inclined structure, “horizontal” is defined with respect to the incline.

Typically, the first temperature is maintained to be greater than, approximately equal to, or equal to wet bulb temperature. Wet-bulb temperature is the lowest temperature that can be reached under current ambient conditions by the evaporation of water only, and is determined by both actual air temperature (or dry-bulb temperature) and humidity. For instance, if the dry-bulb temperature is 40° C., the wet-bulb temperature can range from about 15° C. to about 40° C., depending on the humidity level. In locations where the wet bulb temperature is approximately 30° C., the first temperature is greater than or approximately equal to 30° C. In some embodiments, the first temperature is maintained to be approximately equal to or equal to dry-bulb temperature.

Alternatively, the first temperature is maintained at a desired temperature using, for example, a cooling device (e.g., a chiller) or a natural cooling source (e.g., ocean water, lake water).

In general, the second temperature is maintained to be less than, approximately equal to, or equal to the desired temperature of the culture medium. Typically, the second temperature is within about 5° C. of the desired temperature of the culture medium, which in turn, depends on the temperature conditions useful for culturing phototrophic microorganisms and/or conditions useful for producing carbon-based products of interest, for example, biofuels, using phototrophic microorganisms. Thus, if the desired temperature of the culture medium is 50° C., as is useful for culturing some phototrophic microorganisms, the second temperature is about 45° C. to about 50° C. In some embodiments, the second temperature is maintained to be less than, approximately equal to, or equal to the temperature of the culture medium.

The photobioreactor systems of the invention are useful for culturing both mesophilic and thermophilic phototrophic microorganisms, or mesophiles and thermophiles. Typically, mesophiles are grown in culture medium at a temperature of about 20° C. to about 45° C., preferably, at a temperature of about 25° C. to about 40° C. and, more preferably, at a temperature of about 36° C. to about 38° C. Thermophiles are typically grown in culture medium having a temperature of about 30° C. to about 70° C., preferably, at a temperature of about 45° C. to about 60° C. and, more preferably, at a temperature of about 55° C.

In some embodiments, the first temperature is maintained to be greater than, approximately equal to, or equal to wet bulb temperature and the second temperature is maintained to be less than, approximately equal to, or equal to the desired temperature of the culture medium. In some embodiments, the first temperature is approximately 30° C. and the second temperature is approximately 45° C. However, the difference between the first temperature and the second temperature of horizontally thermally stratified liquid can also be, for example, from about 5° C. to about 25° C., from about 10° C. to about 20° C., or about 15° C.

The values of the first and/or second temperatures can vary, depending on the thermal needs of the system. Thus, the first temperature may be about 30° C. at the start of culture cooling, but may be allowed to increase towards the end of culture cooling during heat storage in order to increase the amount of heat being stored for later use, such as freeze protection or culture warm-up. Conversely, the second temperature may be allowed to decrease in preparation for culture cooling, when the system is less likely to need stored heat.

Generally, the heat storage reservoir is designed to curtail temperature equilibration between the liquid contained in the first end region and the liquid contained in the second end region of the reservoir. Therefore, in some embodiments, the reservoir has a first dimension that is substantially greater than a second dimension. In some embodiments, the first dimension is about 30 to about 300 m, about 100 to about 300 m, about 50 to about 100 m, or about 100 m. In some embodiments, the second dimension is about 5 to about 50 cm, about 5 to about 30 cm, or about 10 to about 15 cm. In some embodiments, the heat storage reservoir has a first dimension of at least about 100 m and a second dimension of about 10 cm to about 30 cm, or a first dimension of at least about 30 m and a second dimension of about 5 cm to about 30 cm. A wide variety of first and second dimensions is possible.

The heat storage reservoir can be made of any material that can be fashioned into a liquid-tight container and is chemically compatible with heat exchange liquid at the temperatures being employed. Typically, the heat storage reservoir is formed of a flexible polymeric material, for example, a flexible composite polymeric material.

The heat storage reservoir can abut the convection chamber. In a particular embodiment, the heat storage reservoir and the convection chamber share a wall. Such an arrangement is depicted, for example, in FIG. 2.

In some embodiments of a photobioreactor system of the invention, the reactor chamber, the convection chamber and the heat storage reservoir are separate chambers of a flexible polymeric capsule, wherein the first and second regions of the heat storage reservoir are connected by a liquid flow path containing thermally stratified heat exchange liquid that increases from the first temperature to the second temperature. In a specific embodiment, the flexible polymeric capsule has a length of at least 50 m. In these embodiments, the capsule can be formed by providing first, second, third, and fourth sheets of flexible polymer film; coupling the first sheet to the second sheet to form a reactor chamber, for example, a reactor chamber comprising a plurality of adjacent channels having substantially longer lengths than widths; coupling the second sheet to the third sheet to form a convection chamber, for example, an un-channeled convection chamber; and coupling the fourth sheet to the third sheet to form the heat storage reservoir, for example, an un-channeled heat storage reservoir.

In other embodiments of a photobioreactor system of the invention, the reactor chamber and the convection chamber are separate chambers of a flexible polymeric capsule, and the heat storage reservoir is free-standing. In these embodiments, the capsule can be formed by providing first, second, and third sheets of flexible polymer film; coupling the first sheet to the second sheet to form a reactor chamber, for example, a reactor chamber comprising a plurality of adjacent channels; and coupling the second sheet to the third sheet to form a convection chamber, for example, an un-channeled convection chamber.

Thus, a reservoir can be an integral part of a photobioreactor capsule or a reservoir can be free-standing (e.g., separate from the capsule).

Examples of heat exchange liquids that can be used in the invention include, but are not limited to, water (e.g., fresh water, salt water), glycol (e.g., ethylene glycol, propylene glycol, or mixtures thereof) and thermal oils.

In general, a photobioreactor system of the invention operates according to the following principles. Heat generated by solar radiation and absorbed by culture medium in a reactor chamber is transferred to the heat exchange liquid or coolant inside the convection chamber. However, instead of immediately rejecting energy in a cooling tower, heat can be stored in the heat storage reservoir in the form of heat exchange liquid, which acts as an energy buffer. The stored heat can then be used later for a variety of purposes. For example, heat rejection can be delayed until a more favorable time of the day/night, or can be spread out over a longer time period, thus significantly reducing the size and cost of the cooling equipment associated with the photobioreactor system. In addition, a substantial fraction of the stored energy can be used to maintain the temperature of the culture medium during non-productive hours, or to provide freeze protection, further reducing the thermal load, operating expenses and the amount of water consumed by an associated cooling system.

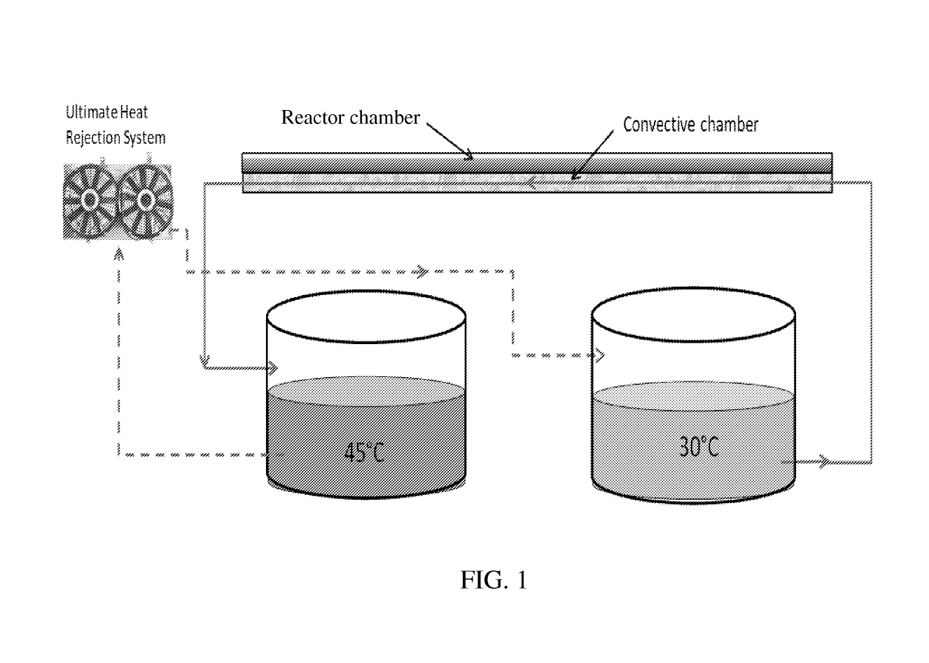

FIG. 1 shows a potential configuration of a photobioreactor system of the invention, wherein the first region and the second region are separate. In this embodiment, the first region and the second region are configured for fluid communication via the convection chamber, but are not in direct fluid communication (in contrast to the first and second regions of the heat storage reservoir depicted in FIG. 2, for example). It is noted that although FIG. 1 illustrates flowing heat exchange liquid from a first region of a heat storage reservoir through a convection chamber and flowing heat exchange liquid from the convection chamber into a second region of the heat storage reservoir, flowing heat exchange liquid from the second region of the heat storage reservoir through the convection chamber and flowing heat exchange liquid from the convection chamber into the first region of the heat storage reservoir is also possible.

FIG. 2 shows another potential configuration of a photobioreactor system of the invention, employing a heat storage reservoir containing horizontally thermally stratified heat exchange liquid. FIG. 2 also illustrates the primary modes of operation of a photobioreactor system of the invention. Although illustrated and described with respect to the photobioreactor system depicted in FIG. 2, the primary modes of operation are generic to the photobioreactor systems of the invention.

In both FIGS. 1 and 2, the reactor chamber is in thermal contact with the convection chamber, allowing for heat exchange between the two. The direction of the energy flow (e.g., solar radiation, heat rejection from heat storage) to and from the culture medium in the reactor chamber changes depending on whether cooling or heating of the culture medium is required. Inclusion of a thermal system provides an opportunity to collect and store thermal energy from incident solar radiation in a liquid form, and subsequently utilize and/or passively reject the stored energy. The modes of operation will be discussed with respect to a mid-summer system operating with a hypothetical microorganism and a desired culture medium temperature of 50° C. A temperature approach between the desired culture medium temperature and the second temperature of about 5° C. or less is likely under most operating conditions. Therefore, the average temperature of the heat exchange liquid in the second region is expected to be about 45° C. or higher in this scenario. The temperature of the heat exchange liquid in the first region is determined by the temperature of the available heat sink, such as wet bulb temperature when evaporative cooling towers are used, or ambient (dry-bulb) temperature when dry cooling towers are used for ultimate heat rejection. For instance, typical mid-summer peak wet bulb temperatures in southwestern states of the U.S. are in the range of low to mid-20's ° C. Assuming an evaporative cooling tower can cool the coolant to within 5° C. of the wet bulb temperature, the first temperature should not exceed about 30° C.

Although particular values of the first and second temperatures are depicted in FIGS. 1 and 2, both temperatures are subject to change, depending on the type of the phototrophic microorganism, geographic location and seasonal variation in ambient conditions. However, the fundamental principles of the thermal management approach described herein and the main modes of the system operation will be very similar regardless of the exact temperature values used for the thermal storage.

A 24-hour operating cycle for a photobioreactor system of the invention is schematically shown in FIG. 2. There are four main modes of operation: 1) morning culture warm-up; 2) idle solar heating; 3) cooling of the photobioreactor system during production; and 4) ultimate heat rejection.

Prior to beginning mode 3 (i.e., at the end of mode 2), the culture medium is below the desired operating temperature (e.g., is less than about 50° C.) and is being heated by incident solar radiation. In the case of two separate thermal storage regions, as depicted in FIG. 1, for example, the second region (e.g., the tank containing heat exchange liquid at 45° C.) is nearly empty while the first region (e.g., the tank containing heat exchange liquid at 30° C.) is nearly full. If the heat storage reservoir is built as a single, stratified thermal storage, most, if not all of the heat storage reservoir contains heat exchange liquid at the first temperature (e.g., 30° C.). When the culture temperature reaches the target value for a given organism, heat exchange liquid of the first temperature is directed from the first region into the convection chamber to prevent overheating of the culture medium and the phototrophic microorganism. The flow rate of the heat exchange liquid can be determined and controlled by a control system. Typically, it is desirable to maintain the temperature of the culture medium throughout the reactor chamber at a desired temperature while minimizing the amount of heat exchange liquid used. In the example shown in FIG. 2, heat exchange liquid is flowed from the first region through the convection chamber (from right to left, as illustrated). Heat is transferred from the culture medium to the heat exchange liquid, so the temperature of the heat exchange liquid as it exits the convection chamber is at the second temperature and approaches the desired culture medium temperature, for example, within approximately 5° C. or less of the desired culture medium temperature. Heat exchange liquid at the second temperature is stored in the second region of the thermal storage reservoir. To minimize the volume of the thermal storage reservoir, or during particularly high solar intensity periods, some fraction of the heat exchange liquid at the second temperature can be sent to an ultimate heat rejection system, such as a dry cooling tower, evaporative cooling tower, geothermal system, cooling pond or other type of cooling system. If some of the heat exchange liquid is sent to the ultimate heat rejection system, it is cooled to the first temperature and is returned to the first region of the heat storage reservoir. This process continues throughout the active solar time of the day, continuously accumulating heat exchange liquid at the second temperature in the second region of the heat storage reservoir. Mode 3 concludes when the culture medium temperature begins to decrease due, for example, to a reduction of solar intensity, at which point further cooling of the culture medium is not needed. At this time, operating mode 4 begins.

During mode 4, the heat exchange liquid at the second temperature, accumulated during mode 3, must be cooled to the first temperature. During most of the year, when ambient conditions permit, accumulated thermal energy can be rejected passively by pumping heat exchange liquid at the second temperature back through the convective chamber (from left to right in FIG. 2), thereby transferring heat to the culture medium and rejecting heat to the environment by means of conduction, convection and/or thermal radiation. The entire solar field (e.g., the surface area of the reactor chamber that collected solar radiation during mode 3) can act as a passive radiator overnight and reject a large fraction of the stored thermal energy. During particularly hot periods, when ambient temperature is relatively high, passive heat rejection will be supplemented by the ultimate heat rejection system, as shown by the (optional) dashed flow paths in FIG. 2. Mode 4 continues until nearly all heat exchange liquid is cooled to the first temperature, except for the heat exchange liquid at the second temperature reserved for operation in mode 1.

Mode 1 is very similar to mode 4, with the minor difference being that flow conditions during mode 4 are designed for maximum heat rejection, whereas flow conditions in mode 1 are designed for maximum increase in pre-dawn culture medium temperature. Several hours of productive operation can potentially be gained daily if culture medium is brought to a desired temperature earlier than otherwise would be possible in a system with no thermal storage. The process continues until culture medium temperature exceeds the second temperature or until there is no heat exchange liquid at the second temperature left in the second region of the thermal storage reservoir.

During mode 2, the culture medium temperature is equal to or exceeds the second temperature, and heat transfer from the heat exchange liquid at the second temperature to the culture medium does not occur. During mode 2, culture medium warm-up to the desired operating temperature occurs due to solar heating. Therefore, the control system monitors culture medium temperature inside the reactor chamber, ready to start the primary cooling mode 3, thus completing a 24-hour cycle. During this time, the flow of the heat exchange liquid can stop and/or heat exchange liquid in the convective chamber may be replaced with a gas to increase the thermal isolation of the reactor chamber, allowing it to heat up faster.

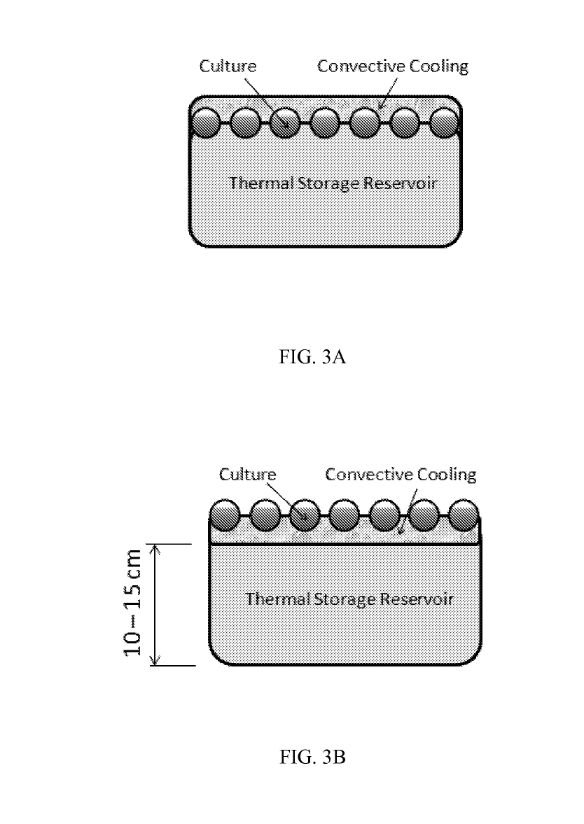

FIGS. 3A and 3B show cross-sections of two potential configurations of a photobioreactor of the invention, wherein the flow of culture medium and heat exchange liquid is perpendicular to the plane of the page. FIG. 3A shows a photobioreactor comprising a reactor chamber comprising a plurality of channels positioned in parallel and having substantially longer lengths than widths for enclosing the phototrophic microorganism and culture medium therefor; and a thermal system including a convection chamber, a heat storage reservoir, and a flow system (not shown), wherein the convection chamber and the reactor chamber are in thermal contact and the reactor chamber and the heat storage reservoir abut. In the embodiment illustrated in FIG. 3A, the entire, or almost the entire, surface area of the reactor chamber is utilized for heat exchange.

FIG. 3B shows a photobioreactor comprising a reactor chamber comprising a plurality of channels positioned in parallel and having substantially longer lengths than widths for enclosing the phototrophic microorganism and culture medium therefor; and a thermal system including a convection chamber, a heat storage reservoir, and a flow system (not shown), wherein the convection chamber and the reactor chamber are in thermal contact. In the embodiment illustrated in FIG. 3B, the heat storage reservoir and the convection chamber abut.

It may be desirable to thermally decouple the culture medium from the heat exchange liquid. In the photobioreactor depicted in FIG. 3A, the culture medium can be thermally decoupled from the heat exchange liquid in the heat storage reservoir by forming or increasing the extent of a gas head space in the heat storage reservoir, thereby reducing thermal contact between the culture medium and the heat exchange liquid in the heat storage reservoir and reducing heat exchange between the culture medium and the heat exchange liquid in the heat storage reservoir.

The photobioreactor represented in FIG. 3B is similar to that depicted in FIG. 2 in that the reactor chamber can be thermally decoupled from the heat storage reservoir by replacing the heat exchange liquid in the convection chamber with gas, such as air, thereby transforming the convection chamber into an insulating barrier and thermally decoupling the culture medium from the heat exchange liquid. To couple or re-couple the reactor chamber and the reservoir, heat exchange liquid can be flowed into and through the convection chamber, establishing or re-establishing convective heating of the culture medium.

Another embodiment of the invention is a method for managing the temperature of culture medium for a phototrophic microorganism in a photobioreactor system of the invention. The method comprises providing a photobioreactor system of the invention, wherein second temperature is greater than the first temperature; and flowing heat exchange liquid from the first or second region of the heat storage reservoir through the flow system into the convection chamber and flowing heat exchange liquid from the convection chamber through the flow system and into the heat storage reservoir, thereby managing the temperature of the culture medium. In some embodiments, for example when it is desirable to maintain or increase the temperature of the culture medium, the method comprises flowing heat exchange liquid from the second region through the flow system and flowing heat exchange liquid from the flow system through the convection chamber to thereby maintain or increase the temperature of the culture medium. Alternatively, when it is desirable to maintain or reduce the temperature of the culture medium, the method comprises flowing heat exchange liquid from the first region through the flow system and flowing heat exchange liquid from the flow system through the convection chamber to thereby maintain or reduce the temperature of the culture medium.

The methods for managing the temperature of culture medium for a phototrophic microorganism in a photobioreactor system of the invention can comprise providing a photobioreactor system of the invention, wherein the second temperature is greater than the first temperature; and flowing heat exchange liquid from the first or second region of the heat storage reservoir through the flow system into the convection chamber and flowing heat exchange liquid from the convection chamber through the flow system and into the second or first region, respectively, of the heat storage reservoir, thereby managing the temperature of the culture medium.

As discussed above with respect to FIGS. 2 and 3B, the culture medium can be thermally decoupled from the heat exchange liquid. Thus, in some embodiments, a method of managing the temperature of culture medium for a phototrophic microorganism in a photobioreactor system further comprises flowing gas through or maintaining gas in the convection chamber, thereby thermally decoupling the reactor chamber from the heat exchange liquid in the convection chamber. Convective heating of the culture medium can be re-established by flowing heat exchange liquid from the first or second region of the heat storage reservoir through the flow system into the convection chamber.

In some embodiments of a photobioreactor system, wherein the heat storage reservoir abuts the convection chamber (e.g., when the reactor chamber, the convection chamber and the heat storage reservoir are separate chambers of a flexible polymeric capsule), the method of managing the temperature of culture medium for a phototrophic microorganism in a photobioreactor system further comprises controlling the extent of a gas head space in the heat storage reservoir to control the thermal contact between the heat exchange liquid in the convection chamber and the heat exchange liquid in the heat storage reservoir. More specifically, controlling the extent of the gas head space comprises forming or increasing the extent of the gas head space in the heat storage reservoir to reduce thermal contact between heat exchange liquid in the convection chamber and heat exchange liquid in the heat storage reservoir, thereby reducing heat exchange between heat exchange liquid in the convection chamber and heat exchange liquid in the heat storage reservoir.

In some embodiments, the flow of the heat exchange liquid through the convection chamber is laminar (e.g., less than about 2,300 Reynolds number or Re, less than about 2,800 Re). Alternatively, the flow of the heat exchange liquid through the convection chamber is transitional flow or turbulent flow (e.g., greater than about 3,000 Re, greater than about 5,000 Re, greater than about 10,000 Re). Thus, in some embodiments of the methods described herein, the method further comprises controlling the flow rate of the heat exchange liquid through the convection chamber.

In some embodiments, the method of managing the temperature of culture medium for a phototrophic microorganism in a photobioreactor system of the invention further comprises managing the amount of heat stored in the reservoir. Managing the amount of heat stored in the reservoir can comprise flowing heat exchange liquid from the second region through a cooling device, and flowing heat exchange liquid from the cooling device into the first region of the heat storage reservoir; or flowing heat exchange liquid from the second region through the flow system and flowing heat exchange liquid from the flow system through the convection chamber under ambient conditions suitable for heat dissipation, thereby reducing the amount of heat stored in the heat storage reservoir. Managing the amount of heat stored in the reservoir can comprise or further comprise flowing heat exchange liquid from the first region through the flow system and flowing heat exchange liquid from the flow system through the convection chamber and into the second region when the temperature of the culture medium is about or exceeds a desired temperature of the culture medium, thereby increasing the amount of heat stored in the heat storage reservoir.

In some embodiments, the method for managing the temperature of the culture medium for a phototrophic microorganism in a photobioreactor system of the invention and/or the method for managing the amount of heat stored in the heat storage reservoir further comprises controlling the difference between the first temperature and the second temperature. For example, a cooling device can be used to maintain the first temperature at any particular desired temperature, thereby controlling the difference between the first temperature and the second temperature.

Thus, in some embodiments, a method for managing the temperature of culture medium for a phototrophic microorganism in a photobioreactor system comprises providing a photobioreactor system of the invention; flowing heat exchange liquid from the first or second region of the heat storage reservoir through the flow system into the convection chamber and flowing heat exchange liquid from the convection chamber through the flow system and into the heat storage reservoir; controlling the difference between the first temperature and the second temperature; and managing the amount of heat stored in the heat storage reservoir, thereby managing the temperature of the culture medium.

Another embodiment of the invention is a method for managing the temperature of a culture medium for a phototrophic microorganism in a photobioreactor system, comprising removing thermal energy from the culture medium to a heat exchange liquid from a first region of a heat storage reservoir to form a heated heat exchange liquid; storing the heated heat exchange liquid in a second region of the heat storage reservoir; removing thermal energy from the heated heat exchange liquid from the second region of the heat storage reservoir to the culture medium, thereby forming a cooled heat exchange liquid; and storing the cooled heat exchange liquid in the first region of the heat storage reservoir.

The methods described herein can further comprise cooling heat exchange liquid from the heat storage reservoir with an ultimate heat rejection system, for example, at times selected to minimize electricity costs.

In preferred embodiments, the methods described herein further comprise flowing the phototrophic microorganism, and culture medium therefor, through the reactor chamber. The flow of culture medium can be laminar, transitional, or turbulent. In a particularly preferred embodiment, the flow of culture medium is turbulent.

### EXEMPLIFICATION

### Example 1

### Heat Transfer in an Energy and Water Efficient Reactor (EWER)

Heat transfer between a coolant and culture medium was evaluated using a multi-chamber reactor capsule comprising a reactor chamber and a convection chamber. Because the energy and water efficient reactor (EWER) concept utilizes thermal storage, it is desirable, for storage size minimization, to reduce the amount of daily coolant use. Therefore, the concept relies on low speed laminar flow heat transfer. It is expected that a heat transfer coefficient of at least 50-55 W/m²-K would allow the reactor to dissipate the maximum expected solar heat flux while keeping the temperature difference between the two ends of the reactor to less than 5° C. In theory, the higher the heat transfer, the smaller the temperature difference between the two ends of the reactor capsule. As a result, a low coolant flow rate is expected to be optimal for heat rejection and thermal storage size minimization.

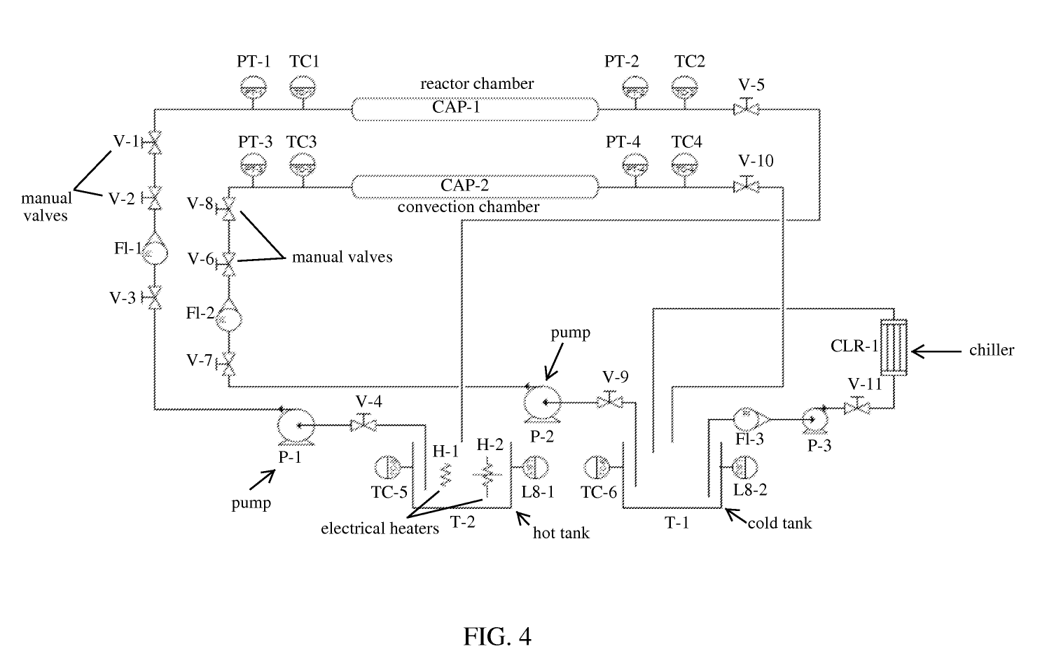

The experimental setup, including a diagram of the piping and instrumentation, is depicted in FIG. 4. The two chambers, CAP-1 and CAP-2, shared a common, thermally-permeable wall. CAP-1 simulates the reactor chamber containing culture medium and is cooled by heat exchange liquid contained in the convection chamber, designated CAP-2. CAP-1 was a four-channeled reactor chamber having a length of about 2.4 m, a width of about 150 mm, and an inflated channel width of about 29 mm. CAP-2 was an un-channeled, single-volume chamber. For initial testing, water was used in both compartments and the overall heat transfer coefficient was measured at different flow velocities in the two chambers. For ease of identification, the water flowed through CAP-1 will be referred to as simulated culture medium, and the water flowed through CAP-2 will be referred to as simulated heat exchange liquid or simulated coolant.

A relatively high temperature was maintained in CAP-1 by two electrical heaters, H-1 and H-2, located inside hot tank T-2. The temperature of the simulated coolant was maintained by circulating water from cold tank T-1 through chiller CLR-1. The flow rates on two sides of the photobioreactor were adjusted by throttling pumps P-1 and P-2 with inlet manual valves V-1 and V-2 on hot side, and V-6 and V-8 on simulated coolant side. The temperature of the simulated culture medium was measured at each end of the reactor chamber by thermocouples TC1 and TC2. The temperature of the simulated coolant was measured at each end of the convection chamber by thermocouples TC3 and TC4.

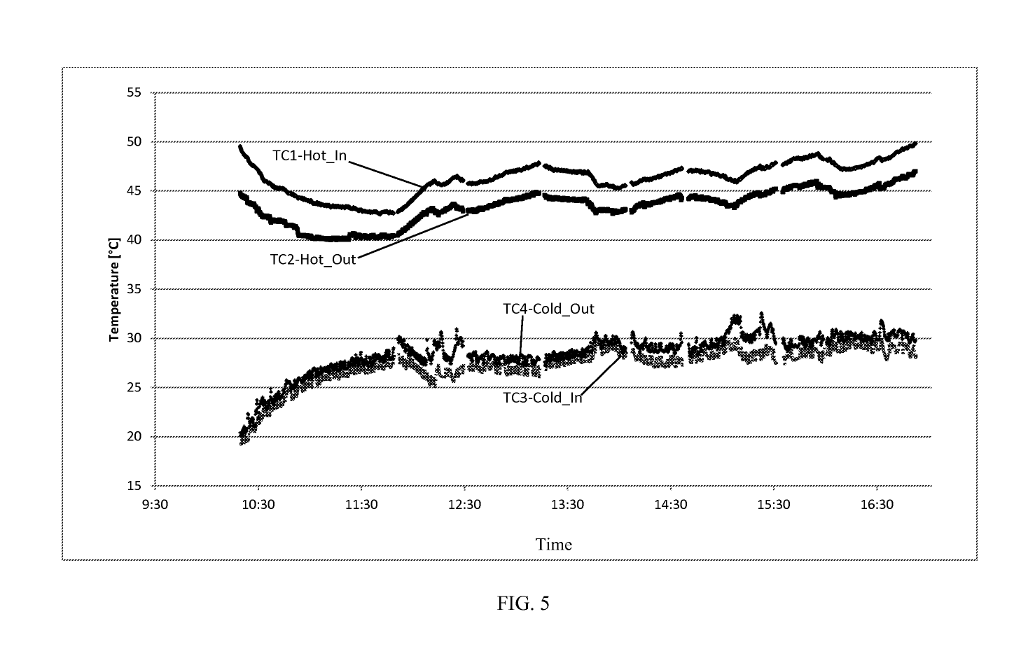

FIG. 5 is a graph of temperature measured at four locations in the test apparatus depicted in FIG. 4 as a function of time.

In a first experiment, simulated culture medium was flowed through the reactor chamber at a Reynolds number approximately equal to Re=12,000 and simulated coolant was flowed through the convection chamber at a variety of Reynolds numbers. Temperatures of the simulated culture medium and simulated coolant were monitored at four locations, indicated by TC1, TC2, TC3 and TC4 in FIG. 4. The heat transfer rate from the simulated culture medium to the simulated coolant was calculated as a product of mass flow rate, fluid heat capacity and the temperature difference between the inlet and outlet of each flow stream. Subsequently, the heat transfer coefficient was estimated as heat transfer rate divided by heat transfer area and divided by log-mean temperature difference between two flow streams.

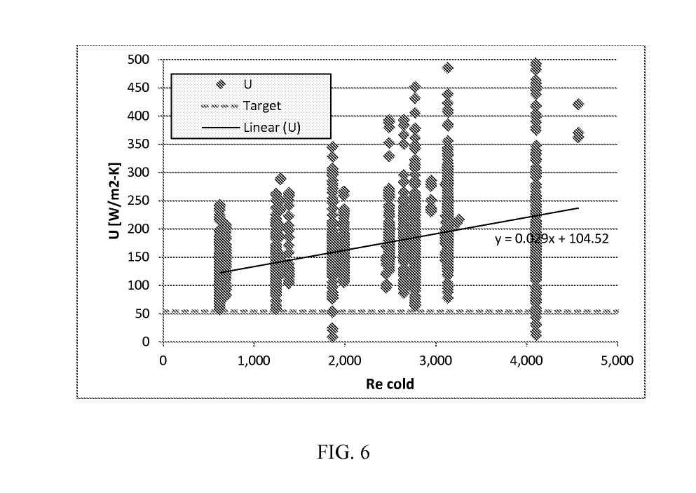

FIG. 6 is a graph of heat transfer coefficient as a function of Reynolds number of the simulated coolant, as measured in the test apparatus depicted in FIG. 4. Nearly all data points satisfied the minimum target value of 50-55 W/m²-K. Of all data points collected, 99.5% are above the 55 W/m²-K threshold value. Table 1 shows the percentage of data points depicted in FIG. 6 above the indicated heat transfer coefficient values.

TABLE 1
Percentage of data points in FIG. 6 above the
indicated heat transfer coefficient (U).

| U, W/m²-K | >55 | >75 | >100 | >150 |
|---|---|---|---|---|
| % data points above U | 99.5% | 95.3% | 89.4% | 57.1% |

The range of calculated heat transfer coefficient values plotted in FIG. 6 for different ranges of Reynolds numbers are shown in Table 2. Over 99% of all recorded data points exceeded the target value of the heat transfer coefficient, and most of the data exceeded the target by a significant margin, proving that the thermal storage concept described herein can be used in conjunction with a reactor chamber formed from thin plastic sheets and thermally coupled to a convection chamber to manage the temperature of culture medium in the reactor chamber.

TABLE 2
Values of heat transfer coefficient (U, W/m²-K)
corresponding to the indicated Reynolds numbers.

| Re range | U min | U max | U average | St. Dev. |
|---|---|---|---|---|
| 600-700 | 57.7 | 242.5 | 116.1 | 40.9 |
| 1200-1400 | 58.3 | 289.2 | 142.5 | 37.5 |
| 1800-2000 | 8.8 | 345.3 | 157.3 | 43.9 |
| 2400-2800 | 63.5 | 451.8 | 184.5 | 60.0 |
| 2900-3300 | 77.9 | 485.5 | 218.7 | 53.1 |
| 4100-4600 | 11.6 | 561.2 | 200.7 | 97.2 |

The teachings of all patents, published applications and references cited herein are incorporated by reference in their entirety.

While this invention has been particularly shown and described with references to example embodiments thereof, it will be understood by those skilled in the art that various changes in form and details may be made therein without departing from the scope of the invention encompassed by the appended claims.

1. A photobioreactor system for a phototrophic microorganism, and culture medium therefor, comprising:
(a) a reactor chamber for enclosing the phototrophic microorganism and culture medium therefor, the reactor chamber being at least partially transparent for light of a wavelength that is photosynthetically active in the phototrophic microorganism; and
(b) a thermal system comprising:
(i) a convection chamber in thermal contact with the reactor chamber and having a first port and a second port;
(ii) a heat storage reservoir having a first region containing a first volume of heat exchange liquid at a first temperature and a second region containing a second volume of heat exchange liquid at a second temperature; and
(iii) a flow system configured for (1) flowing heat exchange liquid from the heat storage reservoir into the first port and through the convection chamber and flowing heat exchange liquid from the convection chamber out of the second port into the heat storage reservoir; and (2) flowing heat exchange liquid from the heat storage reservoir into the second port and through the convection chamber and flowing heat exchange liquid from the convection chamber out of the first port into the heat storage reservoir.

2. (canceled)

3. The photobioreactor system of claim 1, wherein the first and second regions are connected by a liquid flow path containing thermally stratified heat exchange liquid that increases from the first temperature to the second temperature.

4. The photobioreactor system of claim 3, wherein the thermally stratified heat exchange liquid is horizontally thermally stratified heat exchange liquid.

5. (canceled)

6. The photobioreactor system of claim 1, wherein the heat storage reservoir and the convection chamber share a wall.

7. The photobioreactor system of claim 1, wherein the heat storage reservoir has a first dimension of at least 30 m and a second dimension of about 5 cm to about 30 cm.

8. The photobioreactor system of claim 1, wherein the reactor chamber comprises a plurality of channels positioned in parallel and having substantially longer lengths than widths.

9. The photobioreactor system of claim 1, wherein the heat storage reservoir is formed of a flexible polymeric material.

10. A method for managing the temperature of culture medium for a phototrophic microorganism in a photobioreactor system, the method comprising:
providing a photobioreactor system including:
(a) a reactor chamber enclosing the phototrophic microorganism and culture medium therefor, and being at least partially transparent for light of a wavelength that is photosynthetically active in the phototrophic microorganism; and
(b) a thermal system comprising:
(i) a convection chamber in thermal contact with the reactor chamber and having a first port and a second port;
(ii) a heat storage reservoir having a first region containing a first volume of heat exchange liquid at a first temperature and a second region containing a second volume of heat exchange liquid at a second temperature greater than the first temperature; and
(iii) a flow system configured for (1) flowing heat exchange liquid from the heat storage reservoir into the first port and through the convection chamber and flowing heat exchange liquid from the convection chamber out of the second port into the heat storage reservoir; and (2) flowing heat exchange liquid from the heat storage reservoir into the second port and through the convection chamber and flowing heat exchange liquid from the convection chamber out of the first port into the heat storage reservoir; and
flowing heat exchange liquid from the first or second region of the heat storage reservoir through the flow system into the convection chamber and flowing heat exchange liquid from the convection chamber through the flow system and into the heat storage reservoir, thereby managing the temperature of the culture medium.

11. The method of claim 10, comprising:
flowing heat exchange liquid from the second region through the flow system and flowing heat exchange liquid from the flow system through the convection chamber to thereby maintain or increase the temperature of the culture medium; and/or
flowing heat exchange liquid from the first region through the flow system and flowing heat exchange liquid from the flow system through the convection chamber to thereby maintain or reduce the temperature of the culture medium.

12. (canceled)

13. The method of claim 10, further comprising flowing gas through or maintaining gas in the convection chamber, thereby thermally decoupling the reactor chamber from the heat exchange liquid in the convection chamber.

14. The method of claim 10, wherein the first and second regions are connected by a liquid flow path containing thermally stratified heat exchange liquid.

15. The method of claim 14, wherein the thermally stratified heat exchange liquid is horizontally thermally stratified heat exchange liquid.

16-19. (canceled)

20. The method of claim 10, the method further comprising managing the amount of heat stored in the heat storage reservoir, wherein managing the amount of heat stored in the heat storage reservoir comprises:
flowing heat exchange liquid from the second region through a cooling device, and flowing heat exchange liquid from the cooling device into the first region of the heat storage reservoir; or
flowing heat exchange liquid from the second region through the flow system and flowing heat exchange liquid from the flow system through the convection chamber under ambient conditions suitable for heat dissipation,
thereby reducing the amount of heat stored in the heat storage reservoir; and/or
flowing heat exchange liquid from the first region through the flow system and flowing heat exchange liquid from the flow system through the convection chamber and into the second region when the temperature of the culture medium is about or exceeds a desired temperature of the culture medium,
thereby increasing the amount of heat stored in the heat storage reservoir.

21-22. (canceled)

23. The method of claim 10, wherein the flow of the heat exchange liquid through the convection chamber is laminar.

24. The method of claim 10, wherein the first temperature is maintained to be greater than, approximately equal to, or equal to wet bulb temperature.

25. The method of claim 10, wherein the second temperature is maintained to be less than, approximately equal to, or equal to the temperature of the culture medium in the reactor chamber.

26. The method of claim 10, further comprising flowing phototrophic microorganism, and culture medium therefor, through the reactor chamber.

27-28. (canceled)

29. The photobioreactor of claim 1, wherein the reactor chamber and the convection chamber are of substantially equal length and the length is greater than 30 m.

30. A photobioreactor system for a phototrophic microorganism, and culture medium therefor, comprising:
(a) a reactor chamber for enclosing the phototrophic microorganism and culture medium therefor, the reactor chamber being at least partially transparent for light of a wavelength that is photosynthetically active in the phototrophic microorganism and comprising a plurality of adjacent channels having substantially longer lengths than widths; and
(b) a thermal system comprising:
(i) a convection chamber in thermal contact with the reactor chamber and having a first port and a second port;
(ii) a heat storage reservoir abutting the convection chamber and having a first region containing a first volume of heat exchange liquid at a first temperature and a second region containing a second volume of heat exchange liquid at a second temperature; and
(iii) a flow system configured for (1) flowing heat exchange liquid from the heat storage reservoir into the first port and through the convection chamber and flowing heat exchange liquid from the convection chamber out of the second port into the heat storage reservoir; and (2) flowing heat exchange liquid from the heat storage reservoir into the second port and through the convection chamber and flowing heat exchange liquid from the convection chamber out of the first port into the heat storage reservoir,
wherein the reactor chamber, the convection chamber, and the heat storage reservoir are separate chambers of a flexible polymeric capsule having a length of at least 50 m; and the first and second regions are connected by a liquid flow path containing thermally stratified heat exchange liquid that increases from the first temperature to the second temperature.

31. A method for managing the temperature of culture medium for a phototrophic microorganism in a photobioreactor system, the method comprising:
providing the photobioreactor system of claim 30;
flowing heat exchange liquid from the first or second region of the heat storage reservoir through the flow system into the convection chamber and flowing heat exchange liquid from the convection chamber through the flow system and into the heat storage reservoir;
controlling the difference between the first temperature and the second temperature; and
managing the amount of heat stored in the heat storage reservoir,
thereby managing the temperature of the culture medium.

32-36. (canceled)
